(19) United States
(12) Patent Application Publication
(10) Pub. No.: US 2024/0065194 A1
(43) Pub. Date: Feb. 29, 2024

(54) METHOD AND DEVICE FOR MANAGING PHONE NUMBER AND CONTACTS IN WIRELESS COMMUNICATION SYSTEM

(71) Applicant: Samsung Electronics Co., Ltd., Suwon-si (KR)

(72) Inventors: Ngoc Duc NGUYEN, Hanoi City (VN); The Nghia NGUYEN, Hanoi City (VN); Van Khanh NGUYEN, Hanoi City (VN); Thi Tam TRUONG, Hanoi City (VN)

(21) Appl. No.: 18/455,208

(22) Filed: Aug. 24, 2023

(30) Foreign Application Priority Data
Aug. 29, 2022 (KR) 10-2022-0108373

(51) Int. Cl. H04M 1/2757; H04L 65/1104; H04W 12/06

(52) U.S. Cl. CPC H04M 1/2757; H04L 65/1104; H04W 12/068

(57) ABSTRACT

The disclosure relates to a 5G communication system or a 6G communication system for supporting higher data rates beyond a 4G communication system such as long term evolution (LTE). In a wireless communication system, a method performed by a first user equipment (UE) may include: obtaining at least one phone number from a plurality of usable sources, classifying, based on a usage state, the at least one phone number into one of a permanent state, a temporary state, or an invalid state, and transmitting a session initiation protocol (SIP) message including information associated with the at least one classified phone number to a second UE.

### CROSS-REFERENCE TO RELATED APPLICATION

This application is based on and claims priority under 35 U.S.C. § 119 to Korean Patent Application No. 10-2022-0108373, filed on Aug. 29, 2022, in the Korean Intellectual Property Office, the disclosure of which is incorporated by reference herein in its entirety.

### BACKGROUND

### Field

The disclosure relates to a wireless communication system, and for example, to a method and a device for efficiently managing phone numbers and contacts.

### Description of Related Art

Considering the development of wireless communication from generation to generation, the technologies have been developed mainly for services targeting humans, such as voice calls, multimedia services, and data services. Following the commercialization of 5G (5th generation) communication systems, it is expected that the number of connected devices will exponentially grow. Increasingly, these will be connected to communication networks. Examples of connected things may include vehicles, robots, drones, home appliances, displays, smart sensors connected to various infrastructures, construction machines, and factory equipment. Mobile devices are expected to evolve in various form-factors, such as augmented reality glasses, virtual reality headsets, and hologram devices. In order to provide various services by connecting hundreds of billions of devices and things in the 6G (6th generation) era, there have been ongoing efforts to develop improved 6G communication systems. For these reasons, 6G communication systems are referred to as beyond-5G systems.

6G communication systems, which are expected to be commercialized around 2030, will have a peak data rate of tera (1,000 giga)-level bit per second (bps) and a radio latency less than 100 μsec, and thus will be 50 times as fast as 5G communication systems and have the 1/10 radio latency thereof.

In order to accomplish such a high data rate and an ultra-low latency, it has been considered to implement 6G communication systems in a terahertz (THz) band (for example, 95 gigahertz (GHz) to 3 THz bands). It is expected that, due to severer path loss and atmospheric absorption in the terahertz bands than those in mmWave bands introduced in 5G, technologies capable of securing the signal transmission distance (that is, coverage) will become more crucial. It is necessary to develop, as major technologies for securing the coverage, Radio Frequency (RF) elements, antennas, novel waveforms having a better coverage than Orthogonal Frequency Division Multiplexing (OFDM), beamforming and massive Multiple-input Multiple-Output (MIMO), Full Dimensional MIMO (FD-MIMO), array antennas, and multiantenna transmission technologies such as large-scale antennas. In addition, there has been ongoing discussion on new technologies for improving the coverage of terahertz-band signals, such as metamaterial-based lenses and antennas, Orbital Angular Momentum (OAM), and Reconfigurable Intelligent Surface (RIS).

Moreover, in order to improve the spectral efficiency and the overall network performances, the following technologies have been developed for 6G communication systems: a full-duplex technology for enabling an uplink transmission and a downlink transmission to simultaneously use the same frequency resource at the same time; a network technology for utilizing satellites, High-Altitude Platform Stations (HAPS), and the like in an integrated manner; an improved network structure for supporting mobile base stations and the like and enabling network operation optimization and automation and the like; a dynamic spectrum sharing technology via collision avoidance based on a prediction of spectrum usage; an use of Artificial Intelligence (AI) in wireless communication for improvement of overall network operation by utilizing AI from a designing phase for developing 6G and internalizing end-to-end AI support functions; and a next-generation distributed computing technology for overcoming the limit of UE computing ability through reachable super-high-performance communication and computing resources (such as Mobile Edge Computing (MEC), clouds, and the like) over the network. In addition, through designing new protocols to be used in 6G communication systems, developing mechanisms for implementing a hardware-based security environment and safe use of data, and developing technologies for maintaining privacy, attempts to strengthen the connectivity between devices, optimize the network, promote softwarization of network entities, and increase the openness of wireless communications are continuing.

It is expected that research and development of 6G communication systems in hyper-connectivity, including person to machine (P2M) as well as machine to machine (M2M), will allow the next hyper-connected experience. Particularly, it is expected that services such as truly immersive eXtended Reality (XR), high-fidelity mobile hologram, and digital replica could be provided through 6G communication systems. In addition, services such as remote surgery for security and reliability enhancement, industrial automation, and emergency response will be provided through the 6G communication system such that the technologies could be applied in various fields such as industry, medical care, automobiles, and home appliances.

Recently, the number of users who desire to use a user equipment (UE) having one or more phone numbers is increasing. Accordingly, in case that a receiver of a call does not store a phone number of an incoming call in contacts, the receiver may not be aware who the caller is. There is a desire for an operation method for efficiently managing phone numbers and contacts between a caller UE and a receiver UE that operate based on an Internet protocol (IP) multimedia subsystem (IMS) call in a wireless communication system.

### SUMMARY

Embodiments of the disclosure provide a method and device for effectively providing a service in a wireless communication system.

Embodiments of the disclosure provide a method and device for efficiently managing phone numbers and contacts between a caller UE and a receiver UE that operate based on an Internet protocol (IP) multimedia subsystem (IMS) call in a wireless communication system.

In a wireless communication system, according to an example embodiment, a method performed by a first user equipment (UE) may include: obtaining at least one phone number from a plurality of usable sources, classifying, based on a usage state, the at least one phone number into one of a permanent state, a temporary state, or an invalid state, and transmitting a session initiation protocol (SIP) message including information associated with the at least one classified phone number to a second UE.

In a wireless communication system, according to an example embodiment, a method performed by a second user equipment (UE) may include: receiving a session initiation protocol (SIP) message from a first UE, identifying that information associated with a unique phone number of the first UE is not stored, identifying whether the SIP message includes at least one phone number different from the unique phone number of the first UE, and displaying the at least one phone number.

In a wireless communication system, according to an example embodiment, a first user equipment (UE) may include: at least one transceiver and at least one processor functionally coupled to the at least one transceiver, wherein the at least one processor may be configured to: obtain at least one phone number from a plurality of usable sources, classify, based on a usage state, the at least one phone number into one of a permanent state, a temporary state, or an invalid state, and control the transceiver to transmit a session initiation protocol (SIP) message including information associated with the at least one classified phone number to a second UE.

In a wireless communication system, according to an example embodiment, a second user equipment (UE) may include: at least one transceiver and at least one processor functionally coupled to the at least one transceiver, wherein the at least one processor may be configured to receive a session initiation protocol (SIP) message from a first UE via the transceiver, identify that information associated with a unique phone number of the first UE is not stored, identify whether the SIP message includes at least one phone number different from the unique phone number of the first UE, and cause the second UE to display the at least one phone number.

Various example embodiments of the disclosure may provide a method and device for effectively providing a service in a wireless communication system.

Advantageous effects obtainable from the disclosure may not be limited to the above-mentioned effects, and other effects which are not mentioned may be clearly understood, through the following descriptions, by those skilled in the art to which the disclosure pertains.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a diagram illustrating an example of a wireless communication environment according to various embodiments;

FIG. 2A is a block diagram illustrating an example configuration of a base station in a wireless communication system according to various embodiments;

FIG. 2B is a block diagram illustrating an example configuration of a user equipment (UE) in a wireless communication system according to various embodiments;

FIG. 3A is a diagram illustrating an example of an Internet protocol (IP) multimedia subsystem (IMS) communication network system according to various embodiments;

FIG. 3B is a diagram illustrating an example configuration of a UE for Internet protocol (IP) multimedia subsystem (IMS) communication according to various embodiments;

FIG. 4 is a flowchart illustrating an example operation for efficiently managing a phone number and a contact between a caller UE and a receiver UE according to various embodiments;

FIG. 5 is a diagram illustrating an example operation of transferring and managing a phone number and a contact between a caller UE and a receiver UE according to various embodiments;

FIG. 6 is a flowchart illustrating an example operation performed by a caller UE in order to obtain a phone number from a server according to various embodiments;

FIG. 7 is a flowchart illustrating an example operation performed by a caller UE in order to obtain a phone number from a neighboring device according to various embodiments;

FIG. 8 is a flowchart illustrating an example operation in which a receiver UE updates a contact based on a received phone number according to various embodiments;

FIG. 9 is a flowchart illustrating an example operation performed by a receiver UE in order to manage a received phone number and a contact in case that an anonymous number refuse function is activated according to various embodiments of the disclosure;

FIG. 10 is a flowchart illustrating an example operation performed by a receiver UE in order to manage a received phone number and a contact in case that an unstored number refuse function is activated according to various embodiments;

FIG. 11 is a flowchart illustrating an example operation performed by a receiver UE in order to manage a received phone number and a contact in case that a black list is activated according to various embodiments;

FIG. 12 is a flowchart illustrating an example operation performed by a receiver UE in order to manage a phone number and a contact based on a valid or invalid key according to various embodiments; and

FIG. 13 is a flowchart illustrating an example operation performed by a receiver UE in order to manage a phone number and a contact based on permanent or temporary phone number information according to various embodiments.

### DETAILED DESCRIPTION

The terms used in the disclosure are used to describe various example embodiments, and are not intended to limit the disclosure. A singular expression may include a plural expression unless they are definitely different in a context. Unless defined otherwise, all terms used herein, including technical and scientific terms, have the same meaning as those commonly understood by a person skilled in the art to which the disclosure pertains. Such terms as those defined in a generally used dictionary may be interpreted to have the meanings equal to the contextual meanings in the relevant field of art, and are not to be interpreted to have ideal or excessively formal meanings unless clearly defined in the disclosure. In some cases, even the term defined in the disclosure should not be interpreted to exclude embodiments of the disclosure.

Hereinafter, various example embodiments of the disclosure will be described based on an approach of hardware. However, various embodiments of the disclosure include a technology that uses both hardware and software, and thus the various embodiments of the disclosure may not exclude the perspective of software. Furthermore, terms referring to network entities, terms referring to device elements, and the like are illustratively used for the sake of descriptive convenience. Therefore, the disclosure is not limited by the terms as used below, and other terms referring to subjects having equivalent technical meanings may be used.

In the following description, terms referring to parameters related to representation of data (e.g., target object, data time interval, resource level, and data type level), terms referring to network entities, terms referring to device elements (appropriately modified according to the disclosure), and the like are illustratively used for the sake of descriptive convenience. Therefore, the disclosure is not limited by the terms as used below, and other terms referring to subjects having equivalent technical meanings may be used.

In the disclosure, various embodiments will be described using terms employed in various communication standards (e.g., the 3rd generation partnership project (3GPP)), but they are only for the sake of illustration. The embodiments of the disclosure may also be easily applied to other communication systems through modifications.

The technology as described below may be applied to various radio access systems, such as code division multiple access (CDMA), frequency division multiple access (FDMA), time division multiple access (TDMA), orthogonal frequency division multiple access (OFDMA), single carrier frequency division multiple access (SC-FDMA). CDMA may be implemented by radio technology such as universal terrestrial radio access (UTRA) or CDMA2000. TDMA may be implemented by radio technology such as global system for mobile communications (GSM), general packet radio service (GPRS), or enhanced data rates for GSM evolution (EDGE). OFDMA may be implemented by radio technology such as IEEE 802.11 (e.g., Wi-Fi), IEEE 802.16 (e.g., WiMAX), IEEE 802-20, or Evolved UTRA (E-UTRA). To clearly describe the disclosure, the following description is mainly directed to 3GPP NR, but the disclosure is not limited thereto.

As used in the disclosure, the expression “greater than” or “less than” is used to determine whether a specific condition is satisfied or fulfilled, but this is intended only to illustrate an example and does not exclude “greater than or equal to” or “equal to or less than”. A condition indicated by the expression “greater than or equal to” may be replaced with a condition indicated by “greater than”, a condition indicated by the expression “equal to or less than” may be replaced with a condition indicated by “less than”, and a condition indicated by “greater than and equal to or less than” may be replaced with a condition indicated by “greater than and less than”.

In the following description, terms referring to signals, terms referring to channels, terms referring to control information, terms referring to network entities, terms referring to device elements, and the like are illustratively used for the sake of descriptive convenience. Therefore, the disclosure is not limited by the terms as used below, and other terms referring to subjects having equivalent technical meanings may be used.

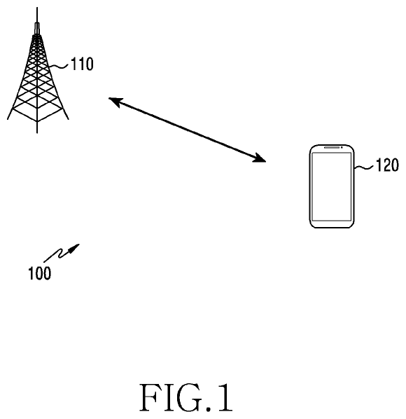

FIG. 1 is a diagram illustrating an example of a wireless communication environment 100 according to various embodiments. FIG. 1 illustrates a base station 110 and a user equipment (UE) 120 as some of the nodes that use wireless channels in a wireless communication system. The UE 120 may be connected to a plurality of base stations. Although not illustrated in FIG. 1, base stations may be connected to the UE 120 via multiple connectivity (e.g., dual connectivity (DC)). According to an embodiment, the UE 120 may perform wireless communication with another UE via a connection to the base station 110. According to various embodiments of the disclosure, wireless communication performed by a caller UE and a receiver UE may include wireless communication performed among a plurality of UEs that is equivalent to or substantially the same as the communication, such as device-to-device (DTD) communication and the like, as well as wireless communication via a base station.

The base station 110 may include a network infrastructure that enables the UE 120 to perform radio access. The base station 110 may have coverage defined by a predetermined geographical area based on a distance in which the base station 110 is capable of delivering a signal. Hereinafter, the term ‘coverage’ used in the descriptions may refer to a service coverage area of the base station 110.

The base station 110 may cover one cell, or may cover multiple cells. Here, the multiple cells may be distinguished based on a frequency that a corresponding cell supports and a sector area that a corresponding cell covers.

The base station 110 may be referred to as an ‘access point (AP)’, an ‘eNodeB (eNB)’, a ‘5^{th} generation node (5G node)’, a ‘5G NodeB (5G NB)’, a ‘next generation node B (gNB)’, a ‘wireless point’, a ‘transmission/reception point (TRP)’, a ‘distributed unit (DU)’, a ‘radio unit (RU)’, a ‘remote radio head (RRH)’, or other terms having the technical meaning equivalent thereto, in addition to a base station. According to various embodiments, the base station 110 may be connected to one or more ‘transmission/reception points (TRP)’. The base station 110 may transmit a downlink signal to the UE 120 or receive an uplink signal from the UE 120 via one or more TRPs.

The UE 120 may be a device used by a user, and may perform communication with the base station 110 via a wireless channel. Depending on the case, the UE 120 may operate, irrespective of handling by a user. That is, at least one of the UEs 120 is a device that performs machine type communication (MTC), and may not be carried by a user. The UE 120 may be referred to as a ‘user equipment (UE)’, a ‘mobile station’, a ‘subscriber station’, a ‘customer-premises equipment (CPE)’, a ‘remote terminal’, a ‘wireless terminal’, an ‘electronic device’, a ‘terminal for a vehicle’, a ‘user device’, or other terms having technical meanings equivalent thereto, in addition a terminal. In addition, according to various embodiments of the disclosure, a UE that makes a call (outgoing call) is referred to as a caller UE, a mobile original (MO), a first UE, or a term equivalent or similar thereto. A UE that receives a call (incoming call) is referred to as a receipt UE, a receiver UE, a mobile terminated (MT), a second UE, or a term equivalent or similar thereto. Here, the MO is a mobile device (or a device) that makes a call to an MT, and the MT is a mobile device (or a device) that receives a call from an MO. Hereinafter, various terms that refer to the above-described UE 120 are interchangeably used in the disclosure.

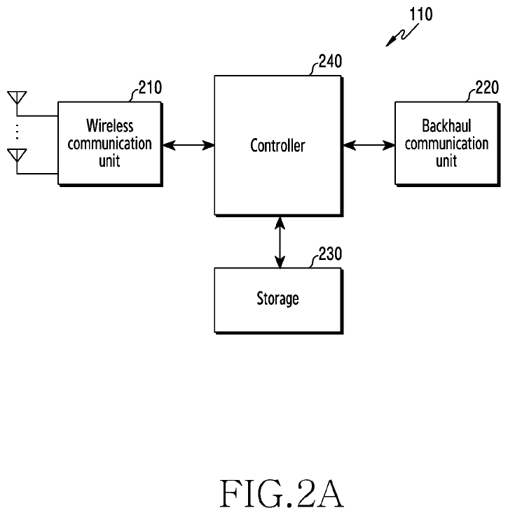

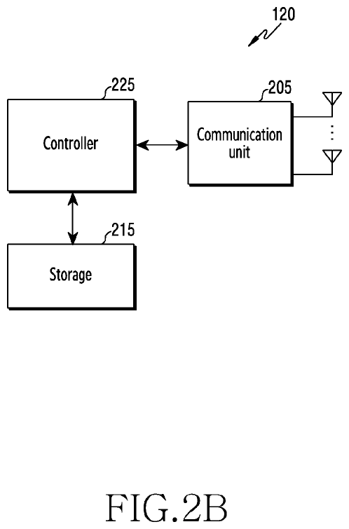

FIG. 2A is a block diagram illustrating an example configuration of a base station in a wireless communication system according to various embodiments. The ending “unit” or “er” used hereinafter may refer to a unit of processing at least one function or operation and may be embodied as hardware, software, or a combination of hardware and software.

Referring to FIG. 2, the base station 110 may include a communication unit (e.g., including communication circuitry) 210, a backhaul communication unit (e.g., including various circuitry) 220, a storage 230, and a controller (e.g., including various processing/control circuitry) 240.

The communication unit 210 may include various communication circuitry performs functions for transmitting or receiving a signal via a wireless channel. For example, the communication unit 210 performs a function of conversion between a baseband signal and a bitstring according to the physical layer standard of a system. For example, in the case of data transmission, the communication unit 210 produces complex symbols by encoding and modulating a transmission bitstring. In addition, in the case of data reception, the communication unit 210 restores a reception bit string by demodulating and decoding a baseband signal. The communication unit 210 may be configured to perform at least one of the operations of a transmission end or a reception end which have are described herein with reference to FIGS. 1 to 10. According to an embodiment, the communication unit 210 may be configured to transmit a downlink signal to the UE 120 or to receive an uplink signal from the UE 120.

The communication unit 210 may up-convert a baseband signal into a radio frequency (RF) band signal and transmit the same via an antenna, and down-converts an RF band signal received via the antenna into a baseband signal. To this end, the communication unit 210 may include a transmission filter, a reception filter, an amplifier, a mixer, an oscillator, a digital-to-analog convertor (DAC), an analog-to-digital convertor (ADC), and the like. In addition, the communication unit 210 may include a plurality of transmission or reception paths. In addition, the communication unit 210 may include at least one antenna array including a plurality of antenna elements. From the perspective of hardware, the communication unit 210 may be configured with a digital unit and an analog unit. The analog unit may include a plurality of sub-units depending on operating power, an operating frequency, or the like. According to an embodiment, the communication unit 210 may include a unit for forming a beam, that is, a beamforming unit. For example, the communication unit 210 may include a massive MIMO unit (MMU) for beamforming.

The communication unit 210 may transmit and receive signals. To this end, the communication unit 210 may include at least one transceiver. For example, the communication unit 210 may transmit a synchronization signal, a reference signal, system information, a message, control information, data, or the like. In addition, the communication unit 210 may perform beamforming. The communication unit 210 may apply a beamforming weight value to a signal desired to be transmitted or received, in order to assign directionality according to configuration by the controller 240. According to an embodiment, the communication unit 210 may produce a baseband signal according to a scheduling result and a transmission power calculation result. In addition, an RF unit in the communication unit 210 may transmit a produced signal via an antenna.

The communication unit 210 may transmit and receive signals as described above. Accordingly, the whole or a part of the communication unit 210 may be referred to as a ‘transmitter’, a ‘receiver’ or a ‘transceiver’. In addition, in the following descriptions, the transmission and reception performed via a wireless channel may include a meaning indicating that the above-described processing is performed by the communication unit 210.

The backhaul communication unit 220 may include various circuitry and provides an interface for performing communication with other nodes in a network. That is, the backhaul communication unit 220 may convert, into a physical signal, a bit string transmitted from the base station 110 to another node, for example, another access node, another base station 110, an upper node, a core network, and the like, and may convert a physical signal received from another node into a bit string.

The storage 230 stores data, such as a basic program for operating the base station 110, an application program, configuration information, and the like. The storage 230 may include a memory. The storage 230 may be embodied as volatile memory, non-volatile memory, or a combination of volatile memory and non-volatile memory. The storage 230 provides data stored therein in response to a request from the controller 240.

The controller 240 may include various processing/control circuitry and controls the overall operation of the base station 110. For example, the controller 240 may transmit and receive signals via the communication unit 210 or the backhaul communication unit 220. Furthermore, the controller 240 records data in the storage 230 and reads data therefrom. The controller 240 may perform the functions of a protocol stack that the communication standard requires. To this end, the controller 240 may include at least one processor. According to various embodiments, the controller 240 may perform control so that the base station 110 performs operations according to various embodiments described below. According to an embodiment, the controller 240 may estimate an uplink channel.

The configuration of the base station 110 illustrated in FIG. 2A is merely an example of a base station, and an example of a base station that performs various embodiments of the disclosure is not limited to the configuration illustrated in FIG. 2A. That is, according to various embodiments, a part of the configuration may be added, removed, or changed.

Although a base station is illustrated as a single entity in FIG. 2A, the disclosure is not limited thereto. The base station 110 according to various embodiments may be embodied to configure an access network having a distributed deployment, as well as an integrated deployment (e.g., an eNB in LTE). In the various example embodiments of FIG. 1 to FIG. 13, a base station may be distinguished as a central unit (CU) and a digital unit (DU), and the CU may be embodied to perform a upper layer (upper layers) function (e.g., packet data convergence protocol (PDCP), RRC), and the DU may be embodied to perform a lower layer (lower layers) function (e.g., medium access control (MAC) or physical (PHY)).

As described above, a base station that has a distributed deployment may further include a configuration for front hall interface communication. According to an embodiment. The base station, as a DU, may perform functions for transmitting or receiving a signal in a wired communication environment. The DU may include a wired interface for controlling a direct connection between devices via a transmission medium (e.g., a copper wire, an optic fiber). For example, the DU may transfer an electric signal to another device via a copper wire, or may perform conversion between an electric signal and an optical signal. The DU may be connected to a CU in a distributed deployment. However, the description is not understood as a meaning that excludes a scenario in which a DU is connected to a CU via a wireless network. In addition, the DU may be connected additionally to a radio unit (RU). However, the description is not understood as a meaning that excludes a wireless environment configured with only a CU and a DU.

FIG. 2B is a block diagram illustrating an example configuration of a UE in a wireless communication system according to various embodiments. The ending “unit” or “er” used hereinafter may refer to a unit of processing at least one function or operation and may be embodied as hardware, software, or a combination of hardware and software.

Referring to FIG. 2B, the UE 120 includes a communication unit (e.g., including communication circuitry) 205, a storage 215, and a controller (e.g., including processing/control circuitry) 225.

The communication unit 205 may include various communication circuitry and performs functions for transmitting or receiving a signal via a wireless channel. For example, the communication unit 205 performs a function of conversion between a baseband signal and a bit string according to the physical layer standard of a system. For example, in the case of data transmission, the communication unit 205 produces complex symbols by encoding and modulating a transmission bit string. In addition, in the case of data reception, the communication unit 205 restores a reception bit string by demodulating and decoding a baseband signal. The communication unit 205 up-converts a baseband signal into an RF band signal and transmits the same via an antenna, and down-converts an RF band signal received via an antenna into a baseband signal. For example, the communication unit 205 may include a transmission filter, a reception filter, an amplifier, a mixer, an oscillator, a DAC, an ADC, and the like.

In addition, the communication unit 205 may include a plurality of transmission or reception paths. In addition, the communication unit 205 may include an antenna unit. The communication unit 205 may include at least one antenna array including a plurality of antenna elements. From the perspective of hardware, the communication unit 205 may include a digital circuit and an analog circuit (e.g., a radio frequency integrated circuit (RFIC)). Here, the digital circuit and the analog circuit may be embodied as a single package. In addition, the communication unit 205 may include a plurality of RF chains. The communication unit 205 may perform beamforming. The communication unit 205 may apply a beamforming weight value to a signal desired to be transmitted or received, in order to assign directionality according to configuration by the controller 225.

In addition, the communication unit 205 may transmit or receive signals. To this end, the communication unit 205 may include at least one transceiver. The communication unit 205 may receive a downlink signal. The downlink signal may include a synchronization signal (SS), a reference signal (RS) (e.g., cell-specific reference signal (CRS) or a demodulation (DM)-RS), system information (e.g., MIB, SIB, or remaining system information (RMSI), or other system information (OSI)), a configuration message, control information, or downlink data, or the like. In addition, the communication unit 205 may transmit an uplink signal. The uplink signal may include uplink control information (UCI), a random access-related signal (e.g., a random access preamble (RAP) (or message 1 (Msg1) or message 3 (Msg3)), or a reference signal (e.g., a sounding reference signal (SRS) or a DM-RS), or the like. For example, uplink control information may include at least one of a scheduling request (SR), ACK/NACK information in a hybrid acknowledge (HARQ) procedure, or channel state information (CSI). According to an embodiment, the communication unit 205 may receive uplink DMRS allocation information. In addition, according to an embodiment, the communication unit 205 may transmit an uplink DMRS symbol.

For example, the communication unit 205 may include an RF processor and a baseband processor. The RF processor may perform a function of transmitting or receiving a signal via a wireless channel, such as band conversion, amplification, and the like of a signal. The RF processor up-converts a baseband signal provided from the baseband processor into an RF band signal and transmits the RF band signal via an antenna, and down-converts an RF band signal received via the antenna into a baseband signal. For example, the RF processor may include a transmission filter, a reception filter, an amplifier, a mixer, an oscillator, a digital to analog convertor (DAC), an analog to digital convertor (ADC), and the like. The UE 120 may include one or more antennas. An RF processor may include a plurality of RF chains. Furthermore, the RF processor may perform beamforming. To perform beamforming, the RF processor may adjust a phase and a size of each signal transmitted or received via a plurality of antennas or antenna elements.

The baseband processor performs a function of conversion between a baseband signal and a bit string according to the physical layer standard of a system. For example, in the case of data transmission, the baseband processor encodes and modulates a transmission bit string so as to produce complex symbols. In addition, in the case of data reception, the baseband processor restores a reception bit string by demodulating and decoding a baseband signal provided from the RF processor. For example, according to an orthogonal frequency division multiplexing (OFDM) scheme, in the case of data transmission, the baseband processor produces complex symbols by encoding and modulating a transmission bit string, maps the complex symbols to subcarriers, and then configures OFDM symbols via an inverse fast Fourier transform (IFFT) operation and cyclic prefix (CP) insertion. Furthermore, in the case of data reception, the baseband processor divides the baseband signal provided from the RF processor in units of OFDM symbols, reconstructs the signals mapped to the subcarriers via a fast Fourier transform (FFT) operation, and then reconstructs a reception bit string via demodulation and decoding.

The communication unit 205 transmits or receives signals as described above. Accordingly, the whole or a part of the communication unit 205 may be referred to as a ‘transmitter’, a ‘receiver’ or a ‘transceiver’. Furthermore, the communication unit 205 may include a plurality of communication modules to support a plurality of different radio access technologies. In addition, the communication unit 205 may include different communication modules for processing signals in different frequency bands. For example, the different radio access technologies may include a wireless LAN (e.g., IEEE 802.1x), a cellular network (e.g., LTE or NR), and the like. Furthermore, the different frequency bands may include a super high frequency (SHF) (e.g., 2.5 GHz or 5 Ghz) band and a millimeter (mm) wave (e.g., 60 GHz) band. In addition, the communication unit 205 may use the same radio access technology in different frequency bands (e.g., a non-licensed band for an NR-unlicensed (NR-U) or a licensed assisted access (LAA), a citizens broadband radio service (e.g., 3.5 GHz)).

The storage 215 may store data, such as a basic program, an application program, configuration information, and the like for operation of the UE 120. The storage 215 may be embodied as volatile memory, non-volatile memory, or a combination of volatile memory and non-volatile memory. The storage 215 may store data, such as a basic program, an application program, configuration information, and the like for operation of the UE 120.

The controller 225 may include various processing/control circuitry and control the overall operation of the UE 120. For example, the controller 225 performs signal transmission and reception via the communication unit 205. In addition, the controller 225 records data in the storage 215, and reads the recorded data therefrom. The controller 225 may perform the functions of a protocol stack that the communication standard requires. To this end, the controller 225 may include at least one processor. The controller 225 may include at least one processor or microprocessor, or may be a part of a processor. A part of the communication unit 205 and the controller 225 may be referred to as a CR The controller 225 may include various modules for performing communication. According to various embodiments, the controller 225 may perform control so that a UE performs operations according to various embodiments described below.

The controller 225 may control general operations of the UE 120. For example, the controller 225 may transmit or receive signals via the communication unit 205. In addition, the controller 225 may record data in the storage 215 and read the recorded data therefrom. To this end, the controller 225 may include at least one processor. For example, the controller 225 may include a communication processor (CP) that performs control for communication, and an application processor (AP) that controls a higher layer such as an application program. According to various embodiments of the disclosure, the controller 225 may be configured to perform a function of sharing a dynamic spectrum. According to an embodiment, the controller 225 may be configured to enable the UE 120 to dynamically use a cell of LTE and a cell of NR in an EN-DC environment. In addition, according to an embodiment, the controller 225 may be configured to enable the UE 120 to dynamically use cells of two nodes in an MR-DC environment, as well as, in the EN-DC environment. In addition, the controller 225 may control the UE 120 to perform operations according to various embodiments described below.

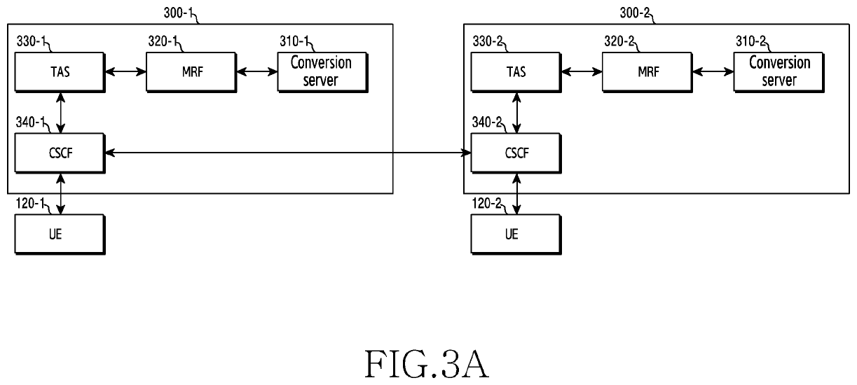

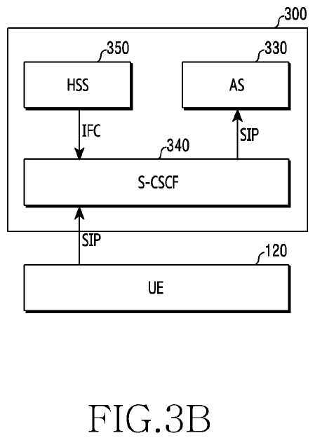

FIG. 3A is a diagram illustrating an example of an Internet protocol (IP) multimedia subsystem (IMS) communication network system according to various embodiments. The IP multimedia subsystem (IMS) refers to a framework to provide a multimedia service in an IP network.

Referring to FIG. 3A, the IMS service network may further include a telephony allocation server (TAS) 330-1 and 330-2 that provides an additional service such as a caller identification service, restriction of an outgoing call/incoming call, switching for an incoming call, or the like, a multimedia resource function (MRF) 320-1 and 320-2 related to a multimedia stream and resource, a conversion service 310-1 and 310-2, and the like.

Referring to FIG. 3A, a UE 120-1 may perform signal transmission or reception with the second UE 120-2 via a first IMS core network system 300-1 that performs signal transmission or reception with the first UE 120-1 and a second IMS core network system 300-2 that performs signal transmission or reception with the second UE 120-2. More particularly, each UE may perform signal transmission or reception with any one IMS core network system 300 among a plurality of IMS core network systems 300, and the first UE 120-1 and the second UE 120-2 may connect a call session via the IMS core network system 300 capable of performing signal transmission or reception with each UE. In this instance, the signal transmission or reception between the IMS core network systems 300 may be performed via a call session control function (CSCF) 340-1 and 340-2 in the IMS core network system 300. Hereinafter, the configuration of a UE for establishing a call session in the IMS system will be described in greater detail.

FIG. 3B is a block diagram illustrating an example configuration of a UE for Internet protocol (IP) multimedia subsystem (IMS) communication according to various embodiments. Referring to FIG. 3B, the major feature of an ALL IP network that replaces all infrastructures of a wireless communication network with an IP is the first 3GPP network that supports an IP multimedia service.

Specifically, referring to FIG. 3B, the feature of an ALL IP network may be implemented by nodes newly added to support an IP multimedia service, and the new nodes may be commonly referred to as the Internet protocol multimedia subsystem (IMS) 300.

The IMS is a set of nodes newly introduced to provide an IP multimedia service, and an IMS service network may include a call session control server (CSCF) 340-1 and 340-2, a home subscriber server (HSS) 350, and an application server (AS) 330. In addition, as illustrated in FIG. 3A, the IMS service network may further include the telephony allocation server (TAS) 330-1 and 330-2 that provides an additional service such as a caller identification service, restriction of an outgoing call/incoming call, switching for an incoming call, or the like, the multimedia resource function (MRF) 320-1 and 320-2 related to a multimedia stream and resource, the conversion service 310-1 and 310-2, and the like. An IMS core network may be configured with a call session control server (CSCF) and a home subscriber server (HSS), and an IMS service network may be configured with an application server (AS).

The call session control server (CSCF) may perform multimedia call control based on a session initiation protocol (SIP). The call session control server (CSCF) 340-1 and 340-2 may include a proxy-call session control function (P-CSCF) (not illustrated), an interrogating-call session control function (I-CSCF) (not illustrated), and a serving-call session control function (S-CSCF) 340.

The home subscriber server (HSS) 350 may store all materials associated with the user equipment (UE) 120 and user data, and may perform functions such as information management, authorization, and the like for management of mobility of a UE and management of a user profile. The home subscriber server (HSS) 350 my briefly provide a home location register/authentication (HLR/AUC) function and an IP multimedia function for an IP multimedia service.

An application server (AS) 330 may be a network object that transfers, to a subscriber, an IP multimedia service including an instant messaging (IM), presence, voice call continuity (VCC), push-to-talk (PTT), voice over Internet protocol (VoIP), a group list management server (GLMS), IMS centralized services (ICS), and the like. For example, the application server 330 may include the TAS 330-1 and 330-2 that provides an additional service such as a caller identification service, restriction of an outgoing call/incoming call, switching for an incoming call, or the like.

In case that the UE 120 transmits a service request message (SIP message) to a P-CSCF for an IP multimedia service, the P-CSCF may transmit the service request message again to an I-CSCF. Based on the service request message, the I-CSCF that receives the service request message may select an S-CSCF 340. The S-CSCF may provide an information-related service to an application server (AS) 330 that is an end point. That is, in case that a predetermined service is provided in an IMS network, the S-CSCF may transfer, to a predetermined application server (AS) 330, an SIP message produced by a UE. According to various embodiments of the disclosure, the SIP message may include an SIP INVITE (or INVITE SIP) message used when initiating a session.

Specifically, referring to FIG. 3B, the home subscriber server (HSS) 350 may be a master database related to a subscriber, and may include basic materials related to a subscriber. The basic materials related to a subscriber may include a subscriber identifier (e.g., number and address information), security information of a subscriber (e.g., network access control information for authentication and authorization), location information of a subscriber (e.g., registration and mobility information of an inter-system level) and service profile information of a subscriber, and the like. In addition, the home subscriber server 350 may operate as a master database of various networks in addition to an IMS network, and may include access information of various communication networks (e.g., 3G, LTE, 5G, and the like) in addition to the IMS network of the subscriber UE 120.

Based on various states of the subscriber UE 120, in addition to information associated with the IMS network itself, the HSS 350 may control transferring of initial filter criteria (IFC) to the S-CSCF 340.

The CSCF may perform functions such as call control, serving profile management, and address processing, and the like. Specifically, the CSCF may include a P-CSCF, an I-CSCF, or the S-CSCF 340. The IMS network 300 may use a session initiation protocol (SIP) as a signaling protocol. Mutual signaling between the subscriber UE 120 and the CSCF, that is, the PCSCF, the I-CSCF, and the S-CSCF, may be performed based on such a session initiation protocol (SIP).

In addition, the S-CSCF 340 may receive an IFC from the HSS 350, together with a subscriber profile, when the subscriber UE 120 is registered with the IMS network 300. The S-CSCF 340 may transfer, to the AS 330, an SIP message produced by the subscriber UE 120 only when a predetermined condition is satisfied. The IFC may include a profile containing a predetermined condition.

The AS 330 may be a network element that transfers, to the subscriber UE 120, an IP multimedia service including an instant messaging (IM), presence, voice call continuity (VCC), push-to-talk (PTT), a voice over internet protocol (VoIP), a group list management server (GLMS), IMS centralized services (ICS), and the like. For example, in case that the AS 330 receives, from the S-CSCF 340, an SIP message produced from the subscriber UE 120, the AS 330 may provide a service corresponding to the SIP message to the subscriber UE 120.

According to various embodiments of the disclosure, there has been provided a description associated with signaling associated with IMS-based call processing performed by a UE or various IMS objects, with reference to FIG. 3A and FIG. 3B. However, the disclosure is not limited thereto, and may be equally applied to call processing signaling among various UEs, in addition to the IMS.

As technology has developed, the number of users of a wireless communication who use one or more phone numbers has been increased. Where a caller attempts to make a call to a call receiver using a predetermined phone number and the receiver does not store, in contacts in advance, the predetermined phone number of the caller, the receiver may not be aware of the caller. As another example, in case that the receiver activates a ‘function of automatically blocking a call with an anonymous number’ or a ‘function of automatically blocking a call with a number that is not stored in contacts’, the UE of the receiver may automatically block a call with a predetermined number that is not stored. As another example, in case that the receiver activates a ‘function of automatically blocking a call with a phone number of a predetermined caller’, and another caller makes a call by borrowing the phone of the predetermined caller who is blocked, the receiver may miss the call from the other caller.

In order to address the above described problem, various embodiments of the disclosure disclose a method and device for obtaining, by a caller or a caller UE, another phone number in addition to a predetermined phone number of the caller UE, and transmitting two or more phone numbers to a receiver UE when attempting to make a call once. In addition, there is disclosed a method and a device for receiving a call from a caller although a receiver or a receiver UE does not store the phone number of a caller UE or a function of blocking the phone number is activated.

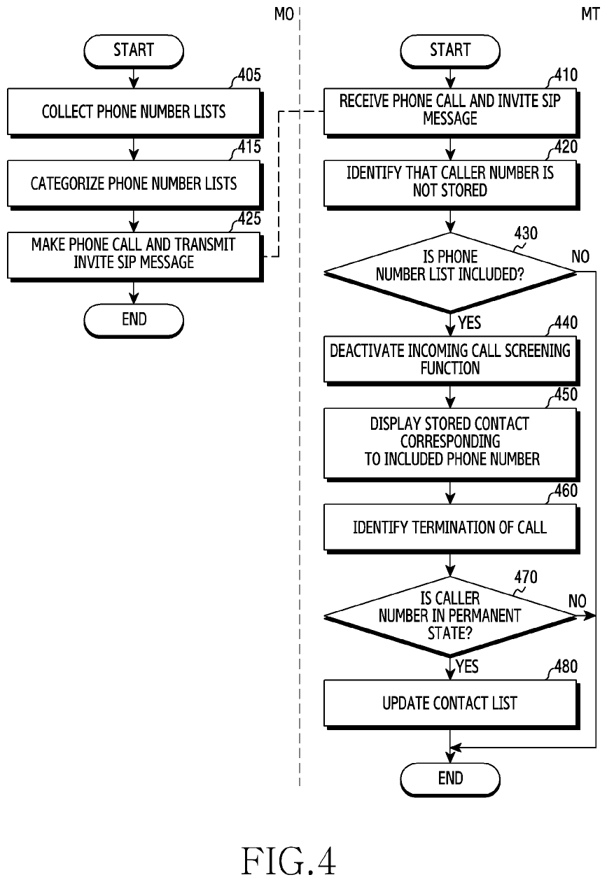

FIG. 4 is a flowchart illustrating an example operation for efficiently managing a phone number and a contact between a caller UE and a receiver UE according to various embodiments. Specifically, FIG. 4A is a flowchart illustrating an example operation for efficiently managing a phone number and a contact by a caller UE (MO) or a receiver UE (MT) according to various embodiments.

In addition, various embodiments of the disclosure may include the whole, part, or a combination of some of the operations performed by a caller UE or a receiver UE. For example, according to an embodiment, irrespective of operations of the receiver UE, performing the whole, part, or a combination of some of the operations of the caller UE may be only included, or the opposite case may also be included. In addition, according to various embodiments of the disclosure, an INVITE SIP message that a caller UE transfers to a receiver UE in FIGS. 4 to 13 may include a message associated with the caller UE, an SIP message, and the like.

Referring to FIG. 4, in operation 405, the caller UE may collect a phone number list. Specifically, the caller UE may obtain one or more phone numbers based on various usable sources. According to an embodiment, the caller UE may obtain one or more phone numbers from at least one of subscriber identity module (SIM) inserted into the caller UE, a cloud server, or a neighboring device. According to an embodiment, the one or more phone numbers that the caller UE obtains may include phone number lists. According to an embodiment, the caller UE may obtain one or more phone numbers from a UE identification object newly installed or inserted from the outside, as well as from the inserted SIM. According to an embodiment, in case that the caller UE attempts to make a call to the receiver UE, the phone number of the caller UE displayed in the receiver UE may be a phone number that is obtained based on the SIM inserted into the caller UE. In addition, hereinafter, according to various embodiments of the disclosure, in case that the caller UE attempts to make a call to the receiver UE, the phone number of the caller UE displayed in the receiver UE may be referred to as a unique phone number (a predetermined phone number or an identification phone number) of the caller UE.

In operation 415, based on the one or more obtained phone numbers, the caller UE may classify (or categorize) the lists of phone numbers. According to an embodiment, the caller UE may classify the lists of phone numbers based on a usage state. A usage state that is one of the criteria for classifying the list of phone numbers may include a permanent state, a temporary state, an invalid state, or the like. For example, the caller UE may identify whether the one or more obtained phone numbers are in the permanent state indicating permanent usage, the temporary state indicating one-time usage, or the invalid state in which a receiver UE is incapable of normally performing reception, and may classify (or categorize), based on the identified state, phone number lists.

In operation 425, the caller UE attempts to make a call to the receiver UE, and may transmit an INVITE SIP message. Specifically, when making a call to the receiver UE, the caller UE may also transmit, based on an IMS network, an INVITE SIP message to the receiver UE. According to an embodiment, the INVITE SIP message that the caller UE transmits may include at least one among information associated with one or more phone numbers obtained by the caller UE, phone number list information, or classified (or categorized) phone number list information. According to an embodiment, the information associated with the one or more phone numbers that the caller UE transmits may include information associated with one or more phone numbers that the caller UE obtains from a plurality of sources, in addition to the unique phone number of the caller UE. According to an embodiment, the classified phone number list information that the caller UE transmits may include information associated with the phone number lists classified based on a usage state.

In operation 410, the receiver UE may receive a call and an INVITE SIP message from the caller UE. According to an embodiment, in case that the receiver UE receives a call from the caller UE, the receiver UE may receive only a call based on the unique phone number of the caller UE or may receive an INVITE SIP message based on an IMS network from the caller UE, together with the call from the caller UE. According to an embodiment, the INVITE SIP message may include at least one among information associated with one or more phone numbers, phone number list information, or classified (or categorized) phone number list information. According to an embodiment, the information associated with the one or more phone numbers may include information associated with one or more phone numbers that the caller UE obtains from a plurality of sources, in addition to the unique phone number of the caller UE. According to an embodiment, the classified phone number list information that the caller UE transmits may include information associated with phone number lists classified based on a usage state.

In operation 420, the receiver UE may identify that the one or more phone numbers received from the caller UE are not stored. According to an embodiment, the receiver UE may receive a call from the caller UE, and may identify whether the unique phone number of the caller UE is a phone number (or an anonymous phone number) that is not stored in the receiver UE (or in contacts in the receiver UE). The receiver UE or the contacts in the receiver UE may include contacts that may be obtained from a receiver UE device, software in the device, or an external server accessible by the receiver UE. According to an embodiment, before receiving a call from the caller UE, the receiver UE may activate a ‘function of automatically blocking a call with an anonymous number’, or a ‘function of automatically blocking a call with a number that is not stored in contacts’. According to an embodiment, in case that the above-described blocking functions are activated and the receiver UE detects that the unique phone number of the caller UE is an anonymous phone number of an unstored phone number, the receiver UE may block a call from the caller UE. According to an embodiment, the operation of blocking a call may include the case of not performing any display or operation in associated with a call from the caller UE on a display or in an application of the receiver UE.

In operation 430, the receiver UE may identify whether phone number list information is received from the caller UE. According to an embodiment, in case that the receiver UE receives a call from the caller UE, the receiver UE may identify whether information including an INVITE SIP message is received together with the unique phone number of the caller UE. According to an embodiment, in case that the receiver UE does not receive the INVITE SIP message, the call from the caller UE may be blocked and a related procedure may be terminated. According to an embodiment, in case that the receiver UE receives a call from the caller UE together with the INVITE SIP message, the method may proceed with operation 440.

In operation 440, the receiver UE may deactivate an incoming call screening function. According to an embodiment, in case that the receiver UE receives the INVITE SIP message from the caller UE, the receiver UE may deactivate a ‘function of automatically blocking a call with an anonymous number’, or a ‘function of automatically blocking a call with a number that is not stored in contacts’. For example, in case that the receiver UE receives, from the caller UE, an INVITE SIP message including information associated with another phone number, in addition to the unique phone number of the caller UE, the receiver UE may disregard the activated blocking functions and may proceed with subsequent operation. According to an embodiment, in case that the ‘function of automatically blocking a call with an anonymous number’ or the ‘function of automatically blocking a call with a phone number not stored in contacts’ is not activated, the receiver UE may not perform operation 440.

In operation 450, the receiver UE may display contacts corresponding to one or more phone numbers included in messages (e.g., an INVITE SIP message or a message including the unique phone number of the caller UE) received from the caller UE. According to an embodiment, in case that one or more contacts corresponding to one or more received phone numbers are stored, the receiver UE may display or provide a phone number that does not correspond to any contact together with the stored contacts. According to an embodiment, in case that contacts corresponding to one or more received phone numbers are not stored, the receiver UE may display or provide all of the one or more received phone numbers.

In operation 460, the receiver UE may identify that a call with the caller UE is terminated. According to an embodiment, operation 460 of identifying the termination of a call may not be performed, and subsequent operations may be performed while a call is being performed.

In operation 470, the receiver UE may identify whether the one or more phone numbers received from the caller UE are in the permanent state. According to an embodiment, based on the classified phone number lists included in the INVITE SIP message received from the caller UE, the receiver UE may identify the usage states of one or more phone numbers. According to an embodiment, in case that the one or more phone numbers received by the receiver UE are classified as permanent state, the method may proceed with operation 480. According to an embodiment, in case that the one or more phone numbers received by the receiver UE are classified as not permanent states, the procedure may be terminated. According to various embodiments of the disclosure, although it is illustrated that the receiver UE operates based on whether one or more received phone numbers are in the permanent state in operation 470, the disclosure is not limited thereto and the criteria of a usage state may include at least one of a permanent state, a temporary state, or an invalid state.

In operation 480, the receiver UE may update contacts that the receiver UE includes. The receiver UE or the contacts in the receiver UE may include contacts that may be obtained from a receiver UE device, software in the device, or an external server accessible by the receiver UE. According to an embodiment, based on the usage states of one or more phone numbers, the receiver UE may determine whether to update contacts or a contact list. According to an embodiment, the receiver UE may store, in contacts in the receiver UE, one or more phone numbers received from the caller UE. According to an embodiment, the operation of updating the contacts by the receiver UE may include an operation of storing one or more phone numbers that are not stored in contacts in the receiver UE. According to an embodiment, the operation of updating contacts by the receiver UE may include an operation of merging a second contact into a first contact in case that the one or more phone numbers received by the receiver UE are not stored in the first contact, but are stored in the second contact.

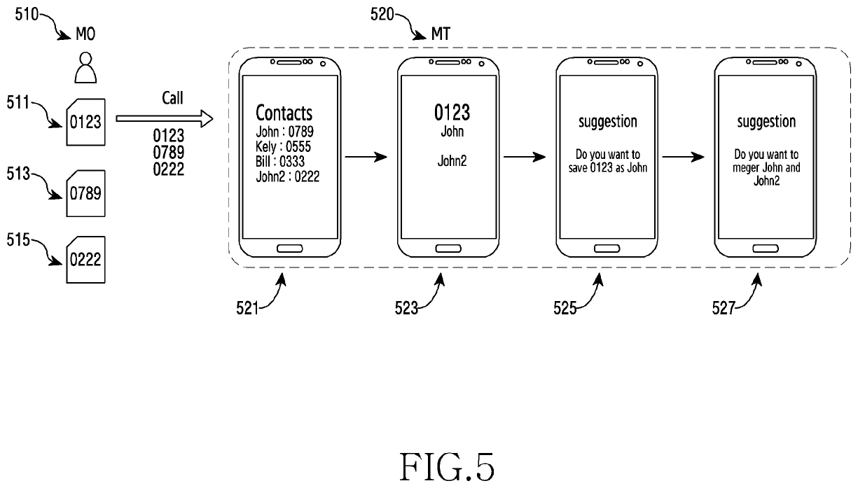

FIG. 5 is a diagram illustrating an example operation of transferring and managing a phone number and a contact between a caller UE and a receiver UE according to various embodiments. Specifically, FIG. 5 visually illustrates some of the various operations according to various embodiments.

The caller UE (or a caller) 510 may have one or more phone numbers 511, 513, and 515 including the unique phone number 511 of the caller UE. According to an embodiment, the caller UE may obtain one or more phone numbers 511, 513, and 515 from various sources including an SIM inserted into the caller UE.

The caller UE may attempt to make a call to the receiver UE (or a receiver) based on one or more phone numbers. According to an embodiment, in case that the caller UE attempts to make a call to the receiver, the unique phone number 511 of the caller UE may be displayed in or provided to the receiver UE. According to an embodiment, the receiver UE 520 may receive an INVITE SIP message including information associated with one or more phone numbers 511, 513, and 515 from the caller UE 510.

The receiver UE 520 may include contacts 521 storing phone numbers. According to an embodiment, one phone number 513 among the one or more phone numbers received from the caller UE 510 may be stored in advance in the contacts 521 in the receiver UE 520.

The receiver UE 520 may display part 511 of the one or more received phone numbers received from the caller UE 510, together with the stored phone number 513 of the caller UE 510 as shown in diagram 523.

The receiver UE 520 may display or provide, to a user, whether to store the part 511 of the one or more phone numbers which are not stored in the contacts and are received from the caller UE 510, as shown in diagram 525.

The receiver UE 520 may display or provide, to the user, whether to merge the contact in which the part 511 of the one or more phone numbers received from the caller UE 510 is stored and the contact in which the phone number of the caller UE 510 is stored in advance, as shown in diagram 527.

Hereinafter, according to various embodiments, each operation that a caller UE or a receiver UE performs will be described in greater detail.

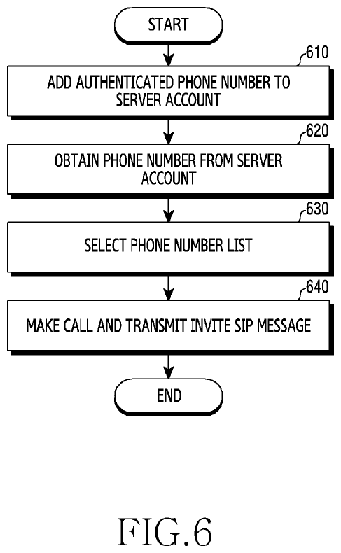

FIG. 6 is a flowchart illustrating an example operation performed by a caller UE in order to obtain a phone number from a server according to various embodiments. Specifically, one or more phone numbers including the unique phone number of the caller UE may be assigned to a caller or the caller UE. In this instance, based on a server account, the caller UE may upload or download one or more phone numbers. According to various embodiments of the disclosure, a series of operations or each operation illustrated in FIG. 6 may be performed in association with or in combination with operations 405 to 425 of FIG. 4.

In operation 610, the caller UE may add an authenticated phone number to the server account. According to an embodiment, the server account of a server connected to the caller UE may include a wireless storage object including a cloud server, a predetermined UE-dedicated server, and the like. According to an embodiment, the caller UE may add (or upload), to the server account, one or more phone numbers allocated to the caller UE. According to an embodiment, the one or more phone numbers that the caller UE adds to the server account may include an authenticated phone number (e.g., including a phone number of which validity is authenticated by the server account), or may include one or more phone numbers obtained from an inserted SIM or a UE identification object that is newly installed or inserted from the outside.

According to an embodiment, the inserted SIM and the server account may be included in the same device. For example, the caller UE may obtain a phone number (or one or more phone numbers) in the inserted SIM, and may add the same to the server account included in the same device.

According to an embodiment, the inserted SIM and the server account may be included in different devices, respectively. For example, a user of the caller UE may attempt to manually add, to the server account, the phone number (or one or more phone numbers) in the inserted SIM. In this instance, the server account may transmit a one-time password (OTP) to the caller UE to which the SIM is inserted, and may request authentication. In case that the caller or the caller UE performs authentication based on the OTP, the phone number (or one or more phone numbers) in the inserted SIM may be added to the server account.

Although not illustrated in FIG. 6, in case that the caller UE adds one or more phone numbers to the server account, the server account may identify or authenticate whether the added phone numbers are valid. According to an embodiment, in case that a predetermined cycle (e.g., one day) elapses after one or more phone numbers are added to the server account, the server account may identify or authenticate whether the phone numbers are valid. For example, the server may obtain, in predetermined cycles, a phone number list from all devices (e.g., a UE) that log on or are connected to the server account. According to an embodiment, in case that one or more phone numbers included in the phone number list that the server account obtains are identified as being usable in all devices that log on or are connected to the server account, the server account may identify the one or more phone numbers are valid. According to an embodiment, the server account may transmit an OTP to a phone number that is not identified as being usable in all devices that log on or are connected to the server account, and may identify whether they are valid. In this instance, the server account may identify that a phone number that is capable of receiving the OTP is valid and may identify that a phone number that is incapable of receiving the OTP is invalid (or a phone number in the invalid state).

In operation 620, the caller UE may obtain a phone number from the server account. According to an embodiment, the phone number that the caller UE obtains from the server account may include one or more phone numbers added in operation 610. According to an embodiment, the one or more phone numbers that the caller UE obtains may include phone number lists. According to an embodiment, the caller UE may store, in a device of the caller UE, one or more phone numbers obtained by logging on the server account.

In operation 630, the caller UE may identify, based on the phone number obtained from the sever account, one or more phone numbers or a phone number list to be transmitted to the receiver UE. According to an embodiment, the one or more phone numbers or the phone number list to be transmitted to the receiver UE may include the unique phone number of the caller UE.

In operation 640, the caller UE may attempt to make a call to the receiver UE, and may transmit an INVITE SIP message. According to an embodiment, the INVITE SIP message that the caller UE transmits to the receiver UE may include information associated with the one or more phone numbers that the caller UE obtains from the server account, together with the unique phone number of the caller UE. According to an embodiment, in case that the caller UE attempts to make a call to the receiver UE, the phone number of the caller UE displayed in the receiver UE may be a phone number that is obtained based on the SIM inserted into the caller UE. In addition, hereinafter, according to various embodiments of the disclosure, in case that the caller UE attempts to make a call to the receiver UE, the phone number of the caller UE displayed in the receiver UE may be referred to as the unique phone number (a predetermined phone number or an identification phone number) of the caller UE.

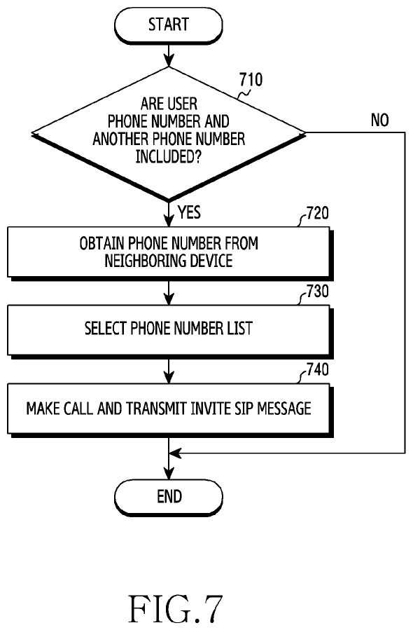

FIG. 7 is a flowchart illustrating an example operation performed by a caller UE in order to obtain a phone number from a neighboring device according to various embodiments. Specifically, one or more phone numbers including the unique phone number of the caller UE may be assigned to a caller or the caller UE. In this instance, the caller UE may transfer one or more phone numbers to another neighboring caller UE device. According to various embodiments, a series of operations or each operation illustrated in FIG. 7 may be performed in association or in combination with operations 405 to 425 of FIG. 4. Hereinafter, referring to FIG. 7, a UE that attempts to make a call to a receiver UE may be referred to as a caller UE, and a UE that is located within a predetermined distance from the caller UE and transfers one or more phone numbers to the caller UE is referred to as another UE.

In operation 710, when attempting to make a call to the receiver UE, the caller UE may identify whether another phone number is included in addition to the unique phone number of the caller UE. For example, the caller UE or a user of the caller UE may identify whether to transmit, to the receiver UE, one or more phone numbers different from a phone number stored in the caller UE or a server related to the caller UE.

In operation 720, the caller UE may receive one or more phone numbers from another UE. According to an embodiment, the other UE may transmit, to the caller UE, one or more phone numbers allocated to the other UE. According to an embodiment, the one or more phone numbers that the other UE transmits to the caller UE may include an authenticated phone number (e.g., including a phone number of which validity is authenticated by a server account) or may include one or more phone numbers obtained from an inserted SIM or a UE identification object that is newly installed or inserted from the outside.

According to an embodiment, in case that it is allowed by the caller UE, the other UE may transmit one or more phone numbers to the caller UE via a short-distance wireless communication technology (e.g., Bluetooth or near field communication (NFC)). For example, via the short-distance wireless communication technology, the caller UE may be connected to the other UE located in a short distance. According to an embodiment, the other UE may transmit information associated with one or more phone numbers to the connected caller UE. According to an embodiment, based on a request received from the connected caller UE, the other UE may transmit information associated with one or more phone numbers to the caller UE.

In operation 730, the caller UE may identify, based on the phone number obtained from the other UE, one or more phone numbers or a phone number list to be transmitted to the receiver UE. According to an embodiment, the one or more phone numbers or the phone number list to be transmitted to the receiver UE may include the unique phone number of the caller UE.

In operation 740, the caller UE attempts to make a call to the receiver UE, and may transmit an INVITE SIP message. According to an embodiment, the INVITE SIP message that the caller UE transmits to the receiver UE may include information associated with the one or more phone numbers that the caller UE obtains from the other UE, together with the unique phone number of the caller UE. According to an embodiment, in case that the caller UE attempts to make a call to the receiver UE, the phone number of the caller UE displayed in the receiver UE may be a phone number that is obtained based on the SIM inserted into the caller UE. In addition, hereinafter, according to various embodiments of the disclosure, in case that the caller UE attempts to make a call to the receiver UE, the phone number of the caller UE displayed in the receiver UE may be referred to as the unique phone number (a predetermined phone number or an identification phone number) of the caller UE.

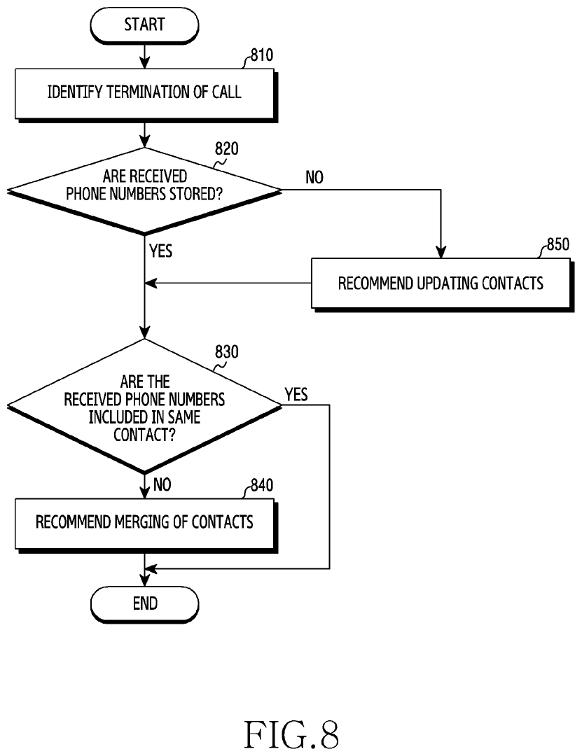

FIG. 8 is a flowchart illustrating an example operation in which a receiver UE updates a contact based on a received phone number according to various embodiments. According to various embodiments, a series of operations or each operation illustrated in FIG. 8 may be performed in association or in combination with operations 470 to 480 of FIG. 4.

In operation 810, the receiver UE may identify that a call with a caller UE is terminated. According to an embodiment, the operation of identifying the termination of a call may not be performed, and subsequent operations may be performed while a call is being performed.

Although not illustrated in FIG. 8, according to various embodiments of the disclosure, subsequent to operation 810, the receiver UE may identify whether one or more phone numbers received from the caller UE are in the permanent state. According to an embodiment, based on classified phone number lists included in the INVITE SIP message received from the caller UE, the receiver UE may identify the usage states of one or more phone numbers. According to an embodiment, in case that the one or more phone numbers that the receiver UE receives are classified as the permanent state, the method may proceed with operation 820. According to an embodiment, in case that the one or more phone numbers that the receiver UE receives are classified as the state different from the permanent state, the procedure may be terminated. According to various embodiments of the disclosure, although it is illustrated that the receiver UE operates based on whether the one or more phone numbers that the receiver UE receives are in the permanent state, the disclosure is not limited thereto and the criteria of a usage state may include at least one of a permanent state, a temporary state, or an invalid state.

In operation 820, the receiver UE may identify whether the one or more received phone numbers are stored in the receiver UE or in contacts in the receiver UE. The receiver UE or the contacts in the receiver UE may include contacts that may be obtained from a receiver UE device, software in the device, or an external server accessible by the receiver UE. According to an embodiment, in case that the one or more phone numbers received by the receiver UE are stored in the receiver UE or in the contacts in the receiver UE, the receiver UE may proceed with operation 830. According to an embodiment, in case that the one or more phone numbers that the receiver UE receives are not stored in the receiver UE or in the contacts in the receiver UE, the receiver UE may proceed with operation 850.

In operation 850, the receiver UE may recommend (e.g., display or provide) updating of contacts included in the receiver UE. According to an embodiment, the receiver UE may determine whether to update contacts or a contact list based on the usage states of the one or more phone numbers. According to an embodiment, the receiver UE may store one or more phone numbers received from the caller UE in the contacts in the receiver UE. According to an embodiment, the operation of updating the contacts by the receiver UE may include an operation of storing one or more phone numbers that are not stored in the contacts in the receiver UE.

In operation 830, the receiver UE may identify whether contacts the stored one or more received phone numbers are the same contact. According to an embodiment, the receiver UE may include contacts that store arbitrary phone numbers. According to an embodiment, the receiver UE may store, as a new contact, one among the one or more phone numbers received from the caller UE, or one of the one or more received phone numbers may be stored in advance in the contacts in the receiver UE. According to an embodiment, in case that contacts respectively corresponding to the one or more phone numbers that the receiver UE receives are the same contact, the procedure may be terminated. According to an embodiment, in case that contacts respectively corresponding to the one or more phone numbers that the receiver UE receives are not the same contact, the procedure may proceed with operation 840.

In operation 840, the receiver UE may recommend (e.g., display or provide) merging of the contacts respectively corresponding to the one or more received phone numbers. According to an embodiment, the receiver UE may display or provide, to a user, whether to merge a contact storing part of the one or more phone numbers received from the caller UE and a contact storing a phone number of the caller UE in advance. According to an embodiment, respective contacts that the receiver UE is capable of merging may be contacts corresponding to the same caller UE.

According to various embodiments of the disclosure, operations 830 to 850 may be performed as a single operation. For example, the operation of updating contacts by the receiver UE may include an operation of merging a second contact into a first contact in case that the one or more phone numbers that the receiver UE receives are not stored in the first contact but are stored in the second contact.

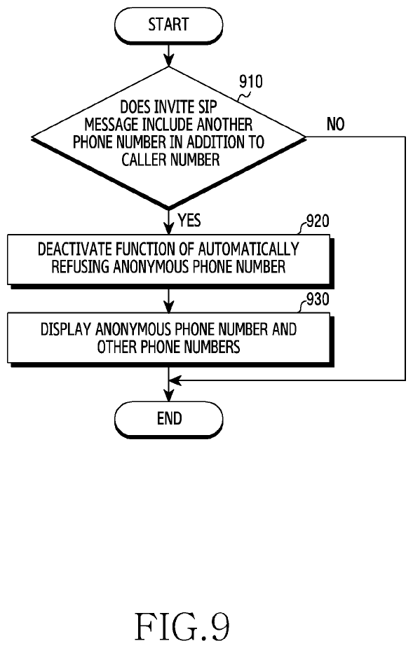

FIG. 9 is a flowchart illustrating an example operation performed by a receiver UE in order to manage a received phone number and a contact in case that an anonymous number refuse function is activated according to various embodiments. According to various embodiments of the disclosure, a series of operations or each operation illustrated in FIG. 9 may be performed in association or in combination with operations 410 to 450 of FIG. 4.

Although not illustrated in FIG. 9, before operation 910, a receiver UE may activate a function of automatically blocking a phone call with an anonymous number. In addition, the unique phone number of a caller UE that attempts to make a call to the receiver UE may be an anonymous number from the perspective of the receiver UE.

In operation 910, the receiver UE may identify whether an INVITE SIP message received from the caller UE includes another phone number in addition to the unique phone number of the caller UE. According to various embodiments of the disclosure, operations that are similar to or substantially the same as operations 410 to 430 of FIG. 4 may be performed.

According to an embodiment, the receiver UE may identify whether information associated with a phone number list is received from the caller UE. According to an embodiment, in case that the receiver UE receives a call from the caller UE, the receiver UE may identify whether information including an INVITE SIP message is received together with the unique phone number of the caller UE. According to an embodiment, in case that the receiver UE does not receive an INVITE SIP message (e.g., including the case in which the INVITE SIP message does not include another phone number in addition to the unique phone number of the caller UE), a call from the caller UE may be blocked and the procedure may be terminated. According to an embodiment, in case that the receiver UE receives a call from the caller UE, together with an INVITE SIP message (e.g., including the case in which the INVITE SIP message includes another phone number in addition to the unique phone number of the caller UE), the method may proceed with operation 920.

In operation 920, the receiver UE may deactivate an incoming call screening function. According to an embodiment, in case that the receiver UE receives an INVITE SIP message from the caller UE, the receiver UE may deactivate a ‘function of automatically blocking a phone call with an anonymous number’. For example, in case that the receiver UE receives, from the caller UE, an INVITE SIP message including information associated with another phone number, in addition to the unique phone number of the caller UE, the receiver UE may disregard the activated blocking functions and may proceed with subsequent operations. According to an embodiment, in case that the ‘function of automatically blocking a call with an anonymous number’ is not activated, the receiver UE may not perform operation 920.

In operation 930, the receiver UE may display contacts corresponding to one or more phone numbers included in messages (e.g., an INVITE SIP message or a message including the unique phone number of the caller UE) received from the caller UE. According to an embodiment, the receiver UE may display or provide one or more phone numbers received together with the unique phone number of the caller UE corresponding to the anonymous number.

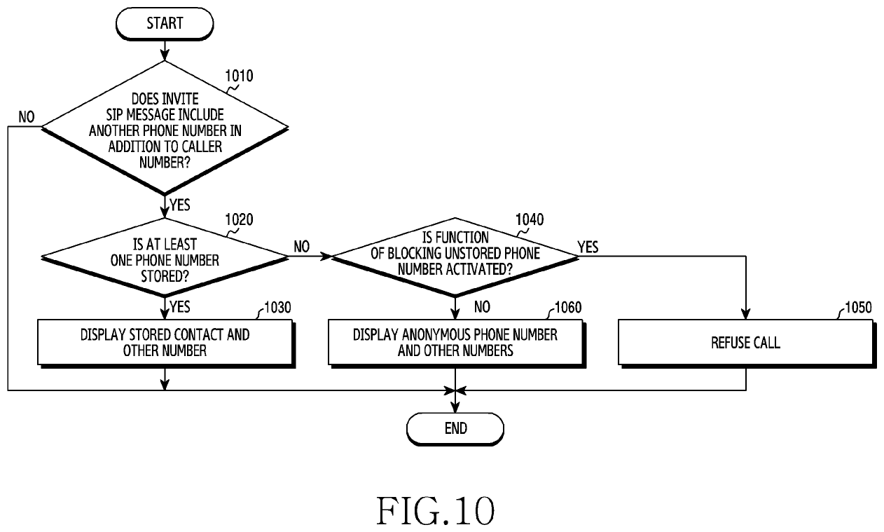

FIG. 10 is a flowchart illustrating an example operation performed by a receiver UE in order to manage a received phone number and a contact in case that an unstored number refuse function is activated according to various embodiments. According to various embodiments of the disclosure, a series of operations or each operation illustrated in FIG. 10 may be performed in association or in combination with operations 410 to 450 of FIG. 4.

Although not illustrated in FIG. 10, before operation 1010, a receiver UE may activate a function of automatically blocking a phone call with a phone number that is not stored in contacts.

In operation 1010, the receiver UE may identify whether an INVITE SIP message received from a caller UE includes another phone number in addition to the unique phone number of the caller UE. According to various embodiments of the disclosure, operations that are similar to or substantially the same as operations 410 to 430 of FIG. 4 may be performed.

According to an embodiment, the receiver UE may identify whether information associated with a phone number list is received from the caller UE. According to an embodiment, in case that the receiver UE receives a call from the caller UE, the receiver UE may identify whether information including an INVITE SIP message is received together with the unique phone number of the caller UE. According to an embodiment, in case that the receiver UE does not receive an INVITE SIP message (e.g., including the case in which the INVITE SIP message does not include another phone number in addition to the unique phone number of the caller UE), a call from the caller UE may be blocked and the procedure may be terminated. According to an embodiment, in case that the receiver UE receives a call from the caller UE, together with an INVITE SIP message (e.g., including the case in which the INVITE SIP message includes another phone number in addition to the unique phone number of the caller UE), the method may proceed with operation 1020.

In operation 1020, the receiver UE may identify whether the one or more phone numbers received from the caller UE are stored. The receiver UE or contacts in the receiver UE may include contacts that may be obtained from a receiver UE device, software in the device, or an external server accessible by the receiver UE. According to an embodiment, in case that part of the one or more phone numbers is stored in the contacts in the receiver UE, the receiver UE may proceed with operation 1030. According to an embodiment, in case that part of the one or more phone numbers is not stored in the contacts in the receiver UE, the receiver UE may proceed with operation 1040.

In operation 1030, the receiver UE may display contacts corresponding to one or more phone numbers included in messages (e.g., an INVITE SIP message or a message including the unique phone number of the caller UE) received from the caller UE. According to an embodiment, in case that one or more contacts corresponding to one or more received phone numbers are stored, the receiver UE may display or provide a phone number that does not correspond to any contact, together with the stored contacts.

In operation 1040, the receiver UE may identify whether a function of automatically blocking a phone call with a phone number that is not stored in contacts is activated. According to an embodiment, in case that the blocking function is activated, the method may proceed with operation 1050. According to an embodiment, in case that the blocking function is not activated, the method may proceed with operation 1060.

In operation 1050, in case that the receiver UE identifies that the unique phone number of the caller UE is an unstored phone number and the blocking function associated therewith is activated, the receiver UE may block a call from the caller UE. According to an embodiment, the operation of blocking a call may include the case of not performing any display or operation in associated with a call from the caller UE on a display or in an application of the receiver UE.

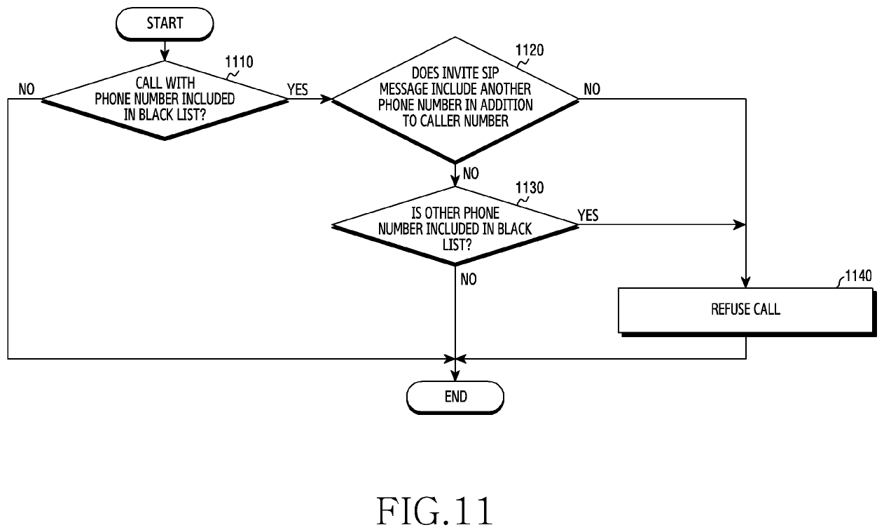

In operation 1060, the receiver UE may display contacts corresponding to one or more phone numbers included in messages (e.g., an INVITE SIP message or a message including the unique phone number of the caller UE) received from the caller UE. According to an embodiment, the receiver UE may display or provide one or more phone numbers received together with the unique phone number of the caller UE corresponding to an anonymous number FIG. 11 is a flowchart illustrating an example operation performed by a receiver UE in order to manage a received phone number and a contact in case that a black list is activated according to various embodiments. According to various embodiments of the disclosure, a series of operations or each operation illustrated in FIG. 11 may be performed in association or in combination with operations 410 to 450 of FIG. 4.

Although not illustrated in FIG. 11, before operation 1110, the receiver UE may produce and store a black list for blocking a call. According to an embodiment, the unique phone number of a caller UE that attempts to make a call to the receiver UE may be included in the black list of the receiver UE.

In operation 1110, the receiver UE may identify whether the unique phone number of the caller UE is included in the black list. According to an embodiment, in case that the unique phone number of the caller UE is included in the black list, the method may proceed with operation 1120. According to an embodiment, in case that the unique phone number of the caller UE is not included in the black list, the procedure may be terminated.

In operation 1120, the receiver UE may identify whether an INVITE SIP message received from the caller UE includes another phone number in addition to the unique phone number of the caller UE. According to various embodiments of the disclosure, operations that are similar to or substantially the same as operations 410 to 430 of FIG. 4 may be performed.

According to an embodiment, the receiver UE may identify whether information associated with a phone number list is received from the caller UE. According to an embodiment, in case that the receiver UE receives a call from the caller UE, the receiver UE may identify whether information including an INVITE SIP message is received together with the unique phone number of the caller UE. According to an embodiment, in case that the receiver UE does not receive an INVITE SIP message (e.g., including the case in which an INVITE SIP message does not include another phone number in addition to the unique phone number of the caller UE), a call from the caller UE may be blocked and the method may proceed with operation 1140. According to an embodiment, in case that the receiver UE receives a call from the caller UE, together with an INVITE SIP message (e.g., including the case in which the INVITE SIP message includes another phone number in addition to the unique phone number of the caller UE), the method may proceed with operation 1130.

In operation 1130, the receiver UE may identify whether one or more other phone numbers in addition to the unique phone number of the caller UE are included in the black list. According to an embodiment, in case that the one or more other phone numbers (e.g., all phone numbers that the receiver UE receives) in addition to the unique phone number of the caller UE that the receiver UE receives are included in the black list, the method may proceed with operation 1140. According to an embodiment, in case that one or more other phone numbers in addition to the unique phone number of the caller UE that the receiver UE receives are not included in the black list, the procedure may be terminated.

In operation 1140, in case that the unique phone number of the caller UE and one or more other phone numbers are all included in the black list, the receiver UE may block a call from the caller UE. According to an embodiment, the operation of blocking a call may include the case of not performing any display or operation in associated with a call from the caller UE on a display or in an application of the receiver UE.

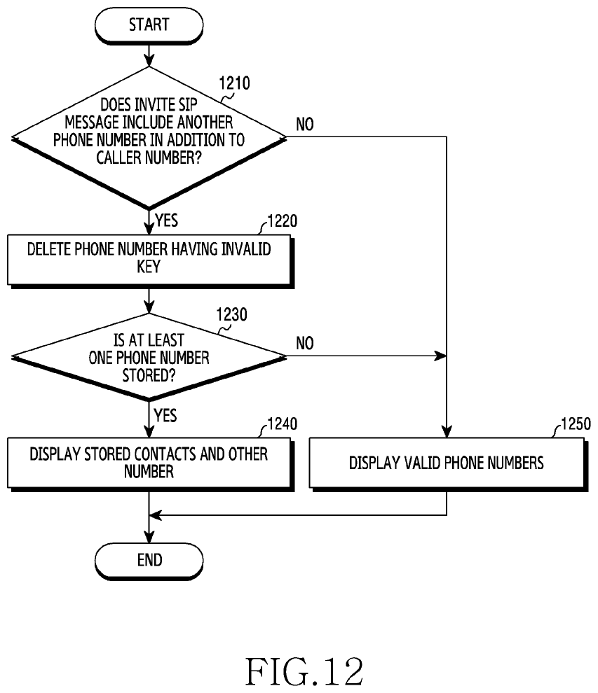

FIG. 12 is a flowchart illustrating an example operation performed by a receiver UE in order to manage a phone number and a contact based on a valid or invalid key according to various embodiments. According to various embodiments of the disclosure, a series of operations or each operation illustrated in FIG. 12 may be performed in association or in combination with operations 410 to 450 of FIG. 4.

Although not illustrated in FIG. 12, before operation 1210, the receiver UE may receive, from a caller UE, an INVITE SIP message further including information associated with one or more phone numbers and keys corresponding thereto, in addition to the unique phone number of a caller UE. According to an embodiment, the key may include personal information such as a hash string produced in the caller UE. According to an embodiment, as the caller UE and the receiver UE perform a call, they may identify and store a key corresponding to the call. Accordingly, a key that each UE stores may be used for identifying information related to the call performed. According to an embodiment, in case that the caller UE attempts to make a call, the receiver UE may receive a current key from the caller UE, and may compare the received current key with a key stored based on a previous call.

Specifically, according to an embodiment, in the case of attempting to make a call to the receiver UE, the caller UE may produce a private key, and may transmit the same to the receiver UE via an INVITE SIP message. According to an embodiment, the key may be a character string such as a header value of a call-identifier (call-ID). According to an embodiment, the receiver UE may store information associated with pairs of one or more phone numbers and keys corresponding thereto, received from the caller UE.

In operation 1210, the receiver UE may identify whether an INVITE SIP message received from the caller UE includes another phone number in addition to the unique phone number of the caller UE. According to various embodiments of the disclosure, operations that are similar to or substantially the same as operations 410 to 430 of FIG. 4 may be performed.

According to an embodiment, the receiver UE may identify whether information associated with a phone number list is received from the caller UE. According to an embodiment, in case that the receiver UE receives a call from the caller UE, the receiver UE may identify whether information including an INVITE SIP message is received together with the unique phone number of the caller UE. According to an embodiment, in case that the receiver UE does not receive an INVITE SIP message (e.g., including the case in which an INVITE SIP message does not include another phone number in addition to the unique phone number of the caller UE), a call from the caller UE may be blocked and the method may proceed with operation 1250. According to an embodiment, in case that the receiver UE receives a call from the caller UE, together with an INVITE SIP message (e.g., including the case in which the INVITE SIP message includes another phone number in addition to the unique phone number of the caller UE), the method may proceed with operation 1220.

In operation 1220, the receiver UE may delete one or more phone numbers having invalid keys. According to an embodiment, one or more phone numbers having invalid keys may include a phone number that does not have a key. According to an embodiment, one or more phone numbers having invalid keys may include a phone number corresponding to a key that is not identical to information associated with keys stored in advance in association with the one or more phone numbers. The receiver UE may identify only one or more phone numbers corresponding to valid keys and may keep security from a received phone number.

In operation 1230, the receiver UE may identify whether the one or more phone numbers received from the caller UE are stored. The receiver UE or contacts in the receiver UE may include contacts that may be obtained from a receiver UE device, software in the device, an external server accessible by the receiver UE. According to an embodiment, in case that part of the one or more phone numbers are stored in the contacts in the receiver UE, the receiver UE may proceed with operation 1240. According to an embodiment, in case that part of the one or more phone numbers is not stored in the contacts in the receiver UE, the receiver UE may proceed with operation 1250.

In operation 1240, the receiver UE may display contacts corresponding to one or more phone numbers included in messages (e.g., an INVITE SIP message or a message including the unique phone number of the caller UE) received from the caller UE. According to an embodiment, in case that one or more contacts corresponding to one or more received phone numbers are stored, the receiver UE may display or provide a phone number that does not correspond to any contact, together with the stored contacts.

In operation 1250, the receiver UE may display a valid phone number among the one or more phone numbers included in messages (e.g., an INVITE SIP message or a message including the unique phone number of the caller UE) received from the caller UE.

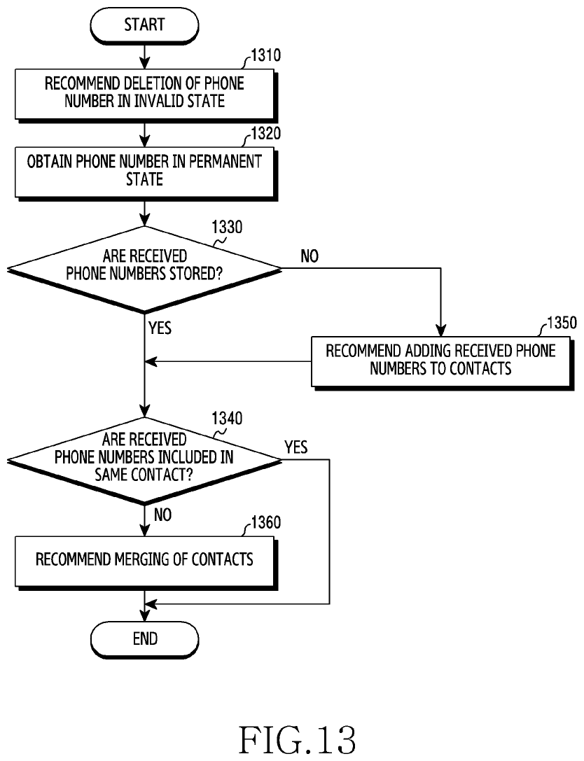

FIG. 13 is a flowchart illustrating an example operation performed by a receiver UE in order to manage a phone number and a contact based on permanent or temporary phone number information according to various embodiments. According to various embodiments of the disclosure, a series of operations or each operation illustrated in FIG. 13 may be performed in association with or in combination with operations 410 to 450 of FIG. 4.

According to an embodiment, although not illustrated in FIG. 13, before operation 1310, a receiver UE may identify whether one or more phone numbers received from a caller UE are in the permanent state. Whether the one or more phone numbers that the receiver UE receives are in the permanent state may be determined via an operation that is similar to or substantially the same as operation 415 of FIG. 4, authentication operation of FIG. 6, or operation 720 of FIG. 7. According to an embodiment, although not illustrated in FIG. 13, before operation 1310, a receiver UE may identify whether one or more phone numbers received from a caller UE are valid. Whether the one or more phone numbers that the receiver UE receives are valid may be determined via operations that are similar to or substantially the same as operation 415 of FIG. 4, authentication operation of FIG. 6, or operation 720 of FIG. 7.

In operation 1310, the receiver UE may recommend deletion of one or more phone numbers having invalid keys. According to an embodiment, one or more phone numbers having invalid keys may include a phone number that does not have a key. According to an embodiment, one or more phone numbers having invalid keys may include a phone number corresponding to a key that is not identical to information associated with keys stored in advance in association with the one or more phone numbers. The receiver UE may identify only one or more phone numbers corresponding to valid keys and may keep security from a phone number received.

In operation 1320, the receiver UE may obtain, from the caller UE, a phone number of the permanent state. According to an embodiment, the receiver UE may identify whether one or more phone numbers received from the caller UE are in the permanent state. According to an embodiment, based on classified phone number lists included in the INVITE SIP message received from the caller UE, the receiver UE may identify the usage states of the one or more phone numbers. According to an embodiment, in case that the one or more phone numbers that the receiver UE receives are classified as the permanent state, the receiver UE may obtain one or more phone numbers in the permanent state.

In operation 1330, the receiver UE may identify whether the one or more received phone numbers are stored in the receiver UE or contacts in the receiver UE. The receiver UE or the contacts in the receiver UE may include contacts that may be obtained from a receiver UE device, software in the device, or an external server accessible by the receiver UE. According to an embodiment, in case that the one or more phone numbers that the receiver UE receives are stored in the receiver UE or in the contacts in the receiver UE, the receiver UE may proceed with operation 1340. According to an embodiment, in case that the one or more phone numbers that the receiver UE receives are not stored in the receiver UE or the contacts in the receiver UE, the receiver UE may proceed with operation 1350.

In operation 1350, the receiver UE may recommend (e.g., display or provide) updating of the contacts included in the receiver UE. According to an embodiment, the receiver UE may determine whether to update the contacts or a contact list based on the usage states of one or more phone numbers. According to an embodiment, the receiver UE may store, in the contacts in the receiver UE, one or more phone numbers received from the caller UE. According to an embodiment, the operation of updating the contacts by the receiver UE may include an operation of storing one or more phone numbers that are not stored in the contacts in the receiver UE.

In operation 1340, the receiver UE may identify whether contacts storing the one or more received phone numbers are the same contact. According to an embodiment, the receiver UE may include contacts in which arbitrary phone numbers are stored. According to an embodiment, the receiver UE may store one phone number among the one or more phone numbers received from the caller UE as a new contact, or one phone number among the one or more received phone numbers may be stored in advance in contacts in the receiver UE. According to an embodiment, in case that contacts respectively corresponding to the one or more phone numbers that the receiver UE receives are the same contact, the procedure may be terminated. According to an embodiment, in case that contacts respectively corresponding to the one or more phone numbers that the receiver UE receives are not the same contact, the procedure may proceed with operation 1360.

In operation 1360, the receiver UE may recommend (e.g., display or provide) merging of the contacts respectively corresponding to the one or more received phone numbers. According to an embodiment, the receiver UE may display or provide, to a user, whether to merge a contact storing part of the one or more phone numbers received from the caller UE and a contact storing a phone number of the caller UE in advance. According to an embodiment, respective contacts that the receiver UE is capable of merging may be contacts corresponding to the same caller UE.

According to various embodiments of the disclosure, operations 1340 to 1360 may be performed as a single operation. For example, the operation of updating contacts by the receiver UE may include an operation of merging a second contact into a first contact in case that the one or more phone numbers received by the receiver UE are not stored in the first contact but are stored in the second contact.

According to various example embodiments of the disclosure, a method performed by a first UE in a wireless communication system may include: obtaining at least one phone number from a plurality of usable sources, classifying, based on a usage state, the at least one phone number into one of a permanent state, a temporary state, or an invalid state, and transmitting a session initiation protocol (SIP) message including information associated with the at least one classified phone number to a second UE.

According to an example embodiment, the method may further include: transmitting, to a server connected to the first UE, a phone number related to the first UE, receiving a phone number authenticated by the server as a valid phone number among the plurality of phone numbers, and transmitting, based on the phone number authenticated as being valid, the SIP message to the second UE.

According to an example embodiment, the phone number authenticated by the server as a valid phone number among the plurality of phone numbers may include a phone number authenticated based on a one-time password (OTP) received from the server.

According to an example embodiment, the method may further include: receiving a plurality of phone numbers from another UE adjacent to the first UE, and transmitting, based on a plurality of phone numbers received from the another UE adjacent to the first UE, the SIP message to the second UE.

According to an example embodiment, the method may further include: producing a key corresponding to the at least one phone number, and transmitting, based on the at least one phone number and the produced key, the SIP message.

According to various example embodiments, a method performed by a second UE in a wireless communication system may include: receiving a session initiation protocol (SIP) message from a first UE, identifying that information associated with a unique phone number of the first UE is not stored, identifying whether the SIP message includes at least one phone number different from the unique phone number of the first UE, and displaying the at least one phone number.

According to an example embodiment, the method may further include deactivating a function of blocking an unstored phone number based on the SIP message including at least one phone number different from the unique phone number of the first UE.

According to an example embodiment, the method may further include: identifying whether the unique phone number of the first UE is included in a black list, identifying whether the at least one phone number is included in the black list based on the SIP message including at least one phone number different from the unique phone number of the first UE, and blocking a call from the first UE based on the at least one phone number being included in the black list.

According to an example embodiment, the method may further include: receiving, from the first UE, the SIP message including the at least one phone number and a key corresponding to the at least one phone number, and deleting, based on the key corresponding to the at least one phone number, a phone number corresponding to an invalid key in the list including the at least one phone number.

According to an example embodiment, the method may further include: updating, based on the at least one phone number, contacts in the second UE based on the SIP message including at least one phone number different from the unique phone number of the first UE.

According to various example embodiments, in a wireless communication system, a first user equipment (UE) may include: at least one transceiver and at least one processor functionally coupled to the at least one transceiver, wherein the at least one processor may be configured to: obtain at least one phone number from a plurality of usable sources, classify, based on a usage state, the at least one phone number into one of a permanent state, a temporary state, or an invalid state, and transmit via the transceiver a session initiation protocol (SIP) message including information associated with the at least one classified phone number to a second UE.

According to an example embodiment, the at least one processor may be further configured to: transmit a phone number related to the first UE to a server connected to the first UE via the transceiver, receive a phone number authenticated by the server as a valid phone number among the plurality of phone numbers, and transmit, based on the phone number authenticated as being valid, the SIP message to the second UE.

According to an example embodiment, the phone number authenticated by the server as a valid phone number among the plurality of phone numbers may include a phone number authenticated based on a one-time password (OTP) received from the server.

According to an example embodiment, the at least one processor may be further configured to: receive a plurality of phone numbers from another UE adjacent to the first UE, and to transmit via the transceiver, based on the plurality of phone numbers received from the another UE adjacent to the first UE, the SIP message to the second UE.

According to an example embodiment, the at least one processor may be further configured to: produce a key corresponding to the at least one phone number, and transmit, based on the at least one phone number and the produced key, the SIP message via the transceiver.

According to various example embodiments, in a wireless communication system, a second user equipment (UE) may include: at least one transceiver and at least one processor functionally coupled to the at least one transceiver, wherein the at least one processor may be configured to: receive a session initiation protocol (SIP) message from a first UE, identify that information associated with a unique phone number of the first UE is not stored, identify whether the SIP message includes at least one phone number different from the unique phone number of the first UE, and cause the second UE to display the at least one phone number.

According to an example embodiment, the at least one processor may be further configured to deactivate a function of blocking an unstored phone number based on the SIP message including at least one phone number different from the unique phone number of the first UE.

According to an example embodiment, the at least one processor may be further configured to: identify whether the unique phone number of the first UE is included in a black list, identify whether the at least one phone number is included in the black list based on the SIP message including at least one phone number different from the unique phone number of the first UE, and block a call from the first UE based on the at least one phone number being included in the black list.

According to an example embodiment, the at least one processor may be further configured to: receive, from the first UE, the SIP message including the at least one phone number and a key corresponding to the at least one phone number, and delete, based on the key corresponding to the at least one phone number, a phone number corresponding to an invalid key in a list including the at least one phone number.

According to an example embodiment, the at least one processor may be further configured to update, based on the at least one phone number, contacts in the second UE based on the SIP message including the at least one phone number different from the unique phone number of the first UE.

The methods according to various embodiments described in the claims or the disclosure may be implemented by hardware, software, or a combination of hardware and software.

When the methods are implemented by software, a computer-readable storage medium for storing one or more programs (software modules) may be provided. The one or more programs stored in the computer-readable storage medium may be configured for execution by one or more processors within the electronic device. The at least one program may include instructions that cause the electronic device to perform the methods according to various embodiments of the disclosure as defined by the appended claims and/or disclosed herein.

The programs (software modules or software) may be stored in non-volatile non-transitory memories including a random access memory and a flash memory, a read only memory (ROM), an electrically erasable programmable read only memory (EEPROM), a magnetic disc storage device, a compact disc-ROM (CD-ROM), digital versatile discs (DVDs), or other type optical storage devices, or a magnetic cassette. Alternatively, any combination of some or all of them may form a memory in which the program is stored. Furthermore, a plurality of such memories may be included in the electronic device.

In addition, the programs may be stored in an attachable storage device which may access the electronic device through communication networks such as the Internet, Intranet, Local Area Network (LAN), Wide LAN (WLAN), and Storage Area Network (SAN) or a combination thereof. Such a storage device may access the electronic device via an external port. Furthermore, a separate storage device on the communication network may access a portable electronic device.

In the above-described example embodiments of the disclosure, an element included in the disclosure is expressed in the singular or the plural according to presented detailed embodiments. However, the singular form or plural form is selected appropriately to the presented situation for the convenience of description, and the disclosure is not limited by elements expressed in the singular or the plural. Therefore, either an element expressed in the plural may also include a single element or an element expressed in the singular may also include multiple elements.

Although specific embodiments have been described in the detailed description of the disclosure, it will be apparent that various modifications and changes may be made thereto without departing from the scope of the disclosure. Therefore, the scope of the disclosure should not be limited to the example embodiments, but should include the appended claims and equivalents thereof. It will also be understood that any of the embodiment(s) described herein may be used in conjunction with any other embodiment(s) described herein.

What is claimed is:

1. A method performed by a first user equipment (UE) in a wireless communication system, the method comprising:
obtaining at least one phone number from a plurality of usable sources;
classifying, based on a usage state, the at least one phone number into one of a permanent state, a temporary state, or an invalid state; and
transmitting a session initiation protocol (SIP) message including information associated with the at least one classified phone number to a second UE.

2. The method of claim 1, further comprising:
transmitting, to a server connected to the first UE, a phone number related to the first UE;
receiving a phone number authenticated by the server as a valid phone number among the plurality of phone numbers; and
transmitting, based on the phone number authenticated as being valid, the SIP message to the second UE.

3. The method of claim 2, wherein the phone number authenticated by the server as a valid phone number among the plurality of phone numbers comprises a phone number authenticated based on a one-time password (OTP) received from the server.

4. The method of claim 1, further comprising:
receiving a plurality of phone numbers from another UE adjacent to the first UE; and
transmitting, based on the plurality of phone numbers received from the another UE adjacent to the first UE, the SIP message to the second UE.

5. The method of claim 1, further comprising:
producing a key corresponding to the at least one phone number; and
based on the at least one phone number and the produced key, transmitting the SIP message.

6. A method performed by a second user equipment (UE) in a wireless communication system, the method comprising:
receiving a session initiation protocol (SIP) message from a first UE;
identifying that information associated with a unique phone number of the first UE is not stored;
identifying whether the SIP message includes at least one phone number different from the unique phone number of the first UE; and
displaying the at least one phone number.

7. The method of claim 6, further comprising deactivating a function of blocking an unstored phone number based on the SIP message including at least one phone number different from the unique phone number of the first UE.

8. The method of claim 6, further comprising:
identifying whether the unique phone number of the first UE is included in a black list;
based on the SIP message including at least one phone number different from the unique phone number of the first UE, identifying whether the at least one phone number is included in the black list; and
based on the at least one phone number being included in the black list, blocking a call from the first UE.

9. The method of claim 6, further comprising:
receiving, from the first UE, the SIP message including the at least one phone number and a key corresponding to the at least one phone number; and
based on the key corresponding to the at least one phone number, deleting a phone number corresponding to an invalid key in a list including the at least one phone number.

10. The method of claim 6, further comprising, based on the SIP message including at least one phone number different from the unique phone number of the first UE, updating, based on the at least one phone number, contacts in the second UE.

11. A first user equipment (UE) in a wireless communication system, the first UE comprising:
a transceiver; and
a controller coupled with the transceiver,
wherein the controller is configured to:
obtain at least one phone number from a plurality of usable sources;
classify, based on a usage state, the at least one phone number into one of a permanent state, a temporary state, or an invalid state; and
transmit a session initiation protocol (SIP) message including information associated with the at least one classified phone number to a second UE.

12. The first UE of claim 11, wherein the controller is further configured to:
transmit a phone number related to the first UE to a server connected to the first UE;
receive a phone number authenticated by the server as a valid phone number among the plurality of phone numbers; and
based on the phone number authenticated as being valid, transmit the SIP message to the second UE.

13. The first UE of claim 12, wherein the phone number authenticated by the server as a valid phone number among the plurality of phone numbers is authenticated based on a one-time password (OTP) received from the server.

14. The first UE of claim 11, wherein the controller is further configured to:
receive a plurality of phone numbers from another UE; and
based on the plurality of phone numbers received from the another UE adjacent to the first UE, transmit the SIP message to the second UE.

15. The first UE of claim 11, wherein the controller is further configured to:
produce a key corresponding to the at least one phone number; and
based on the at least one phone number and the produced key, transmit the SIP message.

16. A second user equipment (UE) in a wireless communication system, comprising:
a transceiver; and
a controller coupled with the transceiver,
wherein the controller is configured to:
receive a session initiation protocol (SIP) message from a first UE;
identify whether information associated with a unique phone number of the first UE is not stored;
identify whether the SIP message includes at least one phone number different from the unique phone number of the first UE; and
display the at least one phone number.

17. The second UE of claim 16, wherein the controller is further configured to deactivate a function of blocking an unstored phone number based on the SIP message including at least one phone number different from the unique phone number of the first UE.

18. The second UE of claim 16, wherein the controller is further configured to:
identify whether the unique phone number of the first UE is included in a black list;
based on the SIP message including at least one phone number different from the unique phone number of the first UE, identify whether the at least one phone number is included in the black list; and
based on the at least one phone number being included in the black list, block a call from the first UE.

19. The second UE of claim 16, wherein the controller is further configured to:
receive, from the first UE, the SIP message including the at least one phone number and a key corresponding to the at least one phone number; and
based on the key corresponding to the at least one phone number, delete a phone number corresponding to an invalid key in a list including the at least one phone number.

20. The second UE of claim 16, wherein the at least one processor is further configured to update, based on the at least one phone number, contacts in the second UE based on the SIP message including the at least one phone number different from the unique phone number of the first UE.
